(19) United States
(12) Patent Application Publication
(10) Pub. No.: US 2016/0095627 A1
(43) Pub. Date: Apr. 7, 2016

(54) SPINAL IMPLANT SYSTEM AND METHOD

(71) Applicant: Warsaw Orthopedic, Inc., Warsaw, IN (US)

(72) Inventor: Hennard M. Michelle, Memphis, TN (US)

(21) Appl. No.: 14/507,223

(22) Filed: Oct. 6, 2014

(51) Int. Cl. A61B 17/70

(52) U.S. Cl. CPC A61B 17/7001; A61B 17/7083; A61B 2017/564

(57) ABSTRACT

A method for treating a spine includes disposing a body including vertebrae in a lateral orientation relative to a surgical fixed surface configured for supporting the body; delivering a plurality of first implants adjacent the vertebrae such that the plurality of first implants are disposed along a sagittal plane of the body; attaching at least one surgical instrument to the plurality of first implants; creating an incision in tissue of the body; spacing the tissue adjacent the incision to define a surgical pathway to the vertebrae along a lateral surgical approach; delivering a second implant along the surgical pathway in a first orientation such that the second implant is aligned with the surgical pathway; and rotating the second implant to a second orientation such that the second implant is aligned with the sagittal plane. Systems and implants are disclosed.

### TECHNICAL HELD

The present disclosure generally relates to medical devices for the treatment of musculoskeletal disorders, and more particularly to a surgical system for implant delivery to a surgical site and a method for treating a spine.

### BACKGROUND

Spinal pathologies and disorders such as scoliosis and other curvature abnormalities, kyphosis, degenerative disc disease, disc herniation, osteoporosis, spondylolisthesis, stenosis, tumor, and fracture may result from factors including trauma, disease and degenerative conditions caused by injury and aging. Spinal disorders typically result in symptoms including deformity, pain, nerve damage, and partial or complete loss of mobility.

Non-surgical treatments, such as medication, rehabilitation and exercise can be effective, however, may fail to relieve the symptoms associated with these disorders. Surgical treatment of these spinal disorders includes fusion, fixation, correction, discectomy, laminectomy and implantable prosthetics. As part of these surgical treatments, spinal constructs, such as, for example, bone fasteners, spinal rods, connectors, plates and interbody devices can be used to provide stability to a treated region. For example, during surgical treatment, surgical instruments can be used to deliver components of the spinal constructs to the surgical site for fixation with bone to immobilize a joint. This disclosure describes an improvement over these prior art technologies.

### SUMMARY

In one embodiment, a method for treating a spine is provided. The method comprises the steps of; disposing a body including vertebrae in a lateral orientation relative to a surgical fixed surface configured for supporting the body; delivering a plurality of first implants adjacent the vertebrae such that the plurality of first implants are disposed along a sagittal plane of the body; attaching at least one surgical instrument to the plurality of first implants; creating an incision in tissue of the body; spacing the tissue adjacent the incision to define a surgical pathway to the vertebrae along a lateral surgical approach; delivering a second implant along the surgical pathway in a first orientation such that the second implant is aligned with the surgical pathway; and rotating the second implant to a second orientation such that the second implant is aligned with the sagittal plane. In some embodiments, implants and systems are disclosed.

### BRIEF DESCRIPTION OF THE DRAWINGS

The present disclosure will become more readily apparent from the specific description accompanied by the following drawings, in which:

FIG. 1 is a plan view of one embodiment of a system in accordance with the principles of the present disclosure disposed with a body;

FIG. 2 is a plan view of one embodiment of a system in accordance with the principles of the present disclosure disposed with a body;

FIG. 3 is a plan view of one embodiment of a system in accordance with the principles of the present disclosure disposed with a body;

FIG. 4 is a plan view of the components of the system and the body shown in FIG. 3; and

FIG. 5 is a plan view of one embodiment of a system in accordance with the principles of the present disclosure disposed with a body.

### DETAILED DESCRIPTION

The exemplary embodiments of the surgical system and related methods of use disclosed are discussed in terms of medical devices for the treatment of musculoskeletal disorders and more particularly, in terms of a surgical system for implant delivery to a surgical site and a method for treating a spine.

In one embodiment, the system is employed with a method for implanting components of the system with a body of a patient when the body is disposed in a lateral position with a surgical fixed surface, such as, for example, a surgical table. In one embodiment, the method includes the step of making an incision to create a surgical pathway in tissue of a patient from a lateral approach. In one embodiment, the method includes the step of providing a surgical instrument, such as, for example, an inserter connected with a spinal rod into an incision such that the spinal rod is oriented perpendicular to a surgical instrument, such as, for example, an extender. In one embodiment, the method includes the step of flipping a spinal rod such that the spinal rod is positioned within the extender.

In some embodiments, the method is used with surgical navigation, such as, for example, fluoroscope or image guidance. In some embodiments, the presently disclosed systems and methods reduce operating time for a surgical procedure and reduce radiation exposure due to fluoroscope or image guidance, for example, by eliminating procedural steps and patient repositioning by implanting system components in one body position.

In one embodiment, one or all of the components of the surgical system are disposable, peel-pack, pre-packed sterile devices. One or all of the components of the surgical system may be reusable. The surgical system may be configured as a kit with multiple sized and configured components.

In one embodiment, the present disclosure may be employed to treat spinal disorders such as, for example, degenerative disc disease, disc herniation, osteoporosis, spondylolisthesis, stenosis, scoliosis and other curvature abnormalities, kyphosis, tumor and fractures. In one embodiment, the present disclosure may be employed with other osteal and bone related applications, including those associated with diagnostics and therapeutics. In some embodiments, the disclosed surgical system and methods may be alternatively employed in a surgical treatment with a patient in a prone, supine position, lateral and/or employ various surgical approaches to the spine, including anterior, posterior, posterior mid-line, direct lateral, postero-lateral, and/or antero-lateral approaches, and in other body regions. The present disclosure may also be alternatively employed with procedures for treating the lumbar, cervical, thoracic, sacral and pelvic regions of a spinal column. The system and methods of the present disclosure may also be used on animals, bone models and other non-living substrates, such as, for example, in training, testing and demonstration.

The present disclosure may be understood more readily by reference to the following detailed description of the embodiments taken in connection with the accompanying drawing figures, which form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, micro discectomy instruments used to remove portions bulging or herniated discs and/or bone spurs, in an effort to alleviate signs or symptoms of the disease or condition. Alleviation can occur prior to signs or symptoms of the disease or condition appearing, as well as after their appearance. Thus, treating or treatment includes preventing or prevention of disease or undesirable condition (e.g., preventing the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

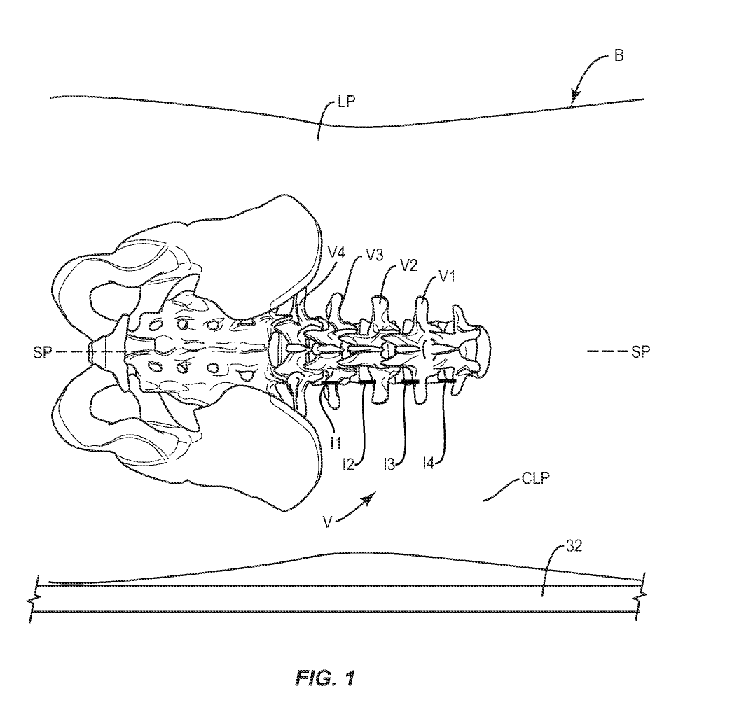

The following discussion includes a description of a surgical system and related methods of employing the surgical system in accordance with the principles of the present disclosure. Alternate embodiments are also disclosed. Reference is made in detail to exemplary embodiments of the present disclosure, which are illustrated in the accompanying figures. Turning to FIGS. 1-5, there are illustrated components of a surgical system, such as, for example, a spinal implant system 10.

The components of spinal implant system 10 can be fabricated from biologically acceptable materials suitable for medical applications, including metals, synthetic polymers, ceramics and bone material and/or their composites, depending on the particular application and/or preference of a medical practitioner. For example, the components of spinal implant system 10, individually or collectively, can be fabricated from materials such as stainless steel alloys, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologix Inc.), thermoplastics such as polyaryletherketone (PAEK) including polyetheretherketone (PEEK), polyetherketoneketone (PEKK) and polyetherketone (PEK), carbon-PEEK composites, PEEK-BaSO₄ polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate such as hydroxyapatite (HA), corraline HA, biphasic calcium phosphate, tricalcium phosphate, or fluorapatite, tri-calcium phosphate (TCP), HA-TCP, calcium sulfate, or other resorbable polymers such as polyaetide, polyglycolide, polytyrosine carbonate, polycaroplaetohe and their combinations, biocompatible ceramics, mineralized collagen, bioactive glasses, porous metals, bone particles, bone fibers, morselized bone chips, bone morphogenetic proteins (BMP), such as BMP-2, BMP-4, BMP-7, rhBMP-2, or rhBMP-7, demineralized bone matrix (DBM), transforming growth factors (TGF, e.g., TGF-β), osteoblast cells, growth and differentiation factor (GDF), insulin-like growth factor 1, platelet-derived growth factor, fibroblast growth factor, or any combination thereof.

Various components of spinal implant system 10 may have material composites, including the above materials, to achieve various desired characteristics such as strength, rigidity, elasticity, compliance, biomechanical performance, durability and radiolucency or imaging preference. The components of spinal implant system 10, individually or collectively, may also be fabricated from a heterogeneous material such as a combination of two or more of the above-described materials. The components of spinal implant system 10 may be monolithically formed, integrally connected or include fastening elements and/or instruments, as described herein.

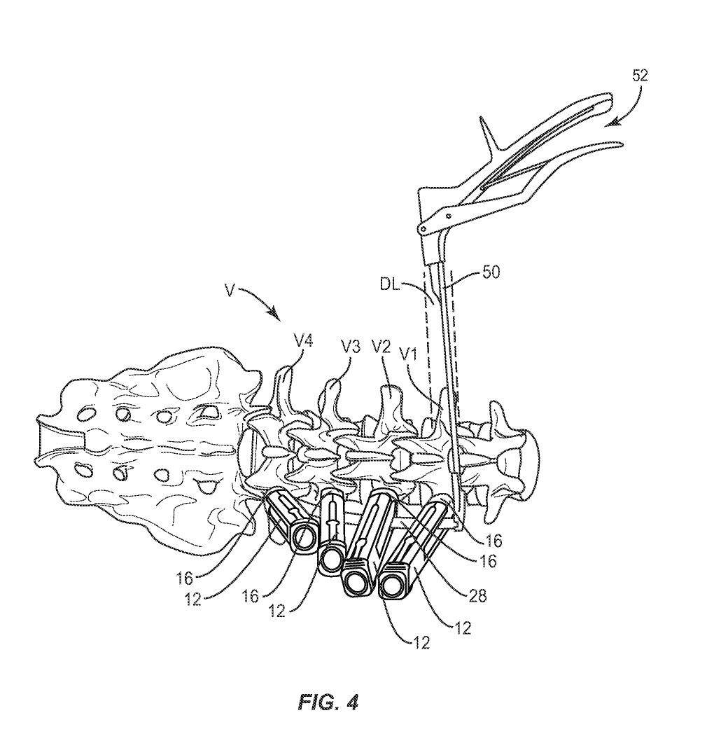

Spinal implant system 10 is employed, for example, with a minimally invasive procedure, including percutaneous techniques, and mini-open surgical techniques to deliver and introduce instrumentation and/or an implant, such as, for example, a bone fastener, at a surgical site within a body B of a patient, which includes, for example, a spine having vertebrae V. Spinal implant system 10 includes extenders 12, as shown in FIG. 4, and fasteners 16, as shown in FIG. 4. Extender 12 is configured for attachment and insertion of a fastener 16 into vertebrae V. In some embodiments, system 10 may comprise various instruments, such as, for example, inserters, reducers, spreaders, distractors, blades, clamps, forceps, elevators and drills, which may be alternately sized and dimensioned, and arranged as a kit.

In some embodiments, one or more of fasteners 16 may be engaged with tissue in various orientations, such as, for example, series, parallel, offset, staggered and/or alternate vertebral levels. In some embodiments, one or more of fasteners 16 may comprise multi-axial screws, sagittal angulation screws, pedicle screws, mono-axial screws, uni-planar screws, facet screws, fixed screws, tissue penetrating screws, conventional screws, expanding screws, wedges, anchors, buttons, caps, snaps, friction fillings, compressive fittings, expanding rivets, staples, nails, adhesives, posts, fixation plates and/or posts.

Each of fasteners 16 comprise a first portion, such as, for example, a receiver and a second portion, such as, for example, an elongated shaft configured for penetrating tissue. The receiver includes a pair of spaced apart arms having an inner surface that defines a U-shaped passageway, One of more of the passageways are configured for disposal of a longitudinal element, such as, for example, a spinal rod 28. In some embodiments, all or only a portion of the passageway may have alternate cross section configurations, such as, for example, oval, oblong, triangular, square, polygonal, irregular, uniform, non-uniform, offset, staggered, and/or tapered. In some embodiments, the arms of the receiver may be disposed at alternate orientations, relative to the shaft, such as, for example, those alternatives described herein.

In one embodiment, each of fasteners 16 have a multi-axial configuration such that the receiver is rotatable to a selected angle through and within an angular range to capture a spinal rod for fixation therein. The inner surface of the receiver includes a thread form configured for engagement with a coupling member, such as, for example, a set screw. The set screw is threaded with the receiver to attach, provisionally fix and/or lock spinal rod 28 with at least one of fasteners 16.

The shaft has a cylindrical cross section configuration and includes an outer surface having an external thread form. In some embodiments, the external thread form may include a single thread turn or a plurality of discrete threads. In some embodiments, other engaging structures may be located on the shaft, such as, for example, a nail configuration, barbs, expanding elements, raised elements and/or spikes to facilitate engagement of the shaft with tissue, such as, for example, vertebrae.

In some embodiments, all or only a portion of the shaft may have alternate cross section configurations, such as, for example, oval, oblong, triangular, square, polygonal, irregular, uniform, non-uniform, offset, staggered, undulating, arcuate, variable and/or tapered. In some embodiments, the outer surface of the shaft may include one or a plurality of openings. In some embodiments, all or only a portion of the outer surface of the shaft may have alternate surface configurations, such as, for example, smooth and/or surface configurations to enhance fixation with tissue, such as, for example, rough, arcuate, undulating, porous, semi-porous, dimpled, polished and/or textured. In some embodiments, all or only a portion of the shaft may be cannulated.

Spinal rod 28 has a cylindrical cross section configuration. In some embodiments, system 10 may include one or a plurality of spinal rods, which may be relatively disposed in a side by side, irregular, uniform, non-uniform, offset and/or staggered orientation or arrangement. In some embodiments, spinal rod 28 can have a uniform thickness/diameter. In some embodiments, spinal rod 28 may have various surface configurations, such as, for example, rough, threaded for connection with surgical instruments, arcuate, undulating, dimpled, polished and/or textured. In some embodiments, the thickness defined by spinal rod 28 may be uniformly increasing or decreasing, or have alternate diameter dimensions along its length. In some embodiments, spinal rod 28 may have various cross section configurations, such as, for example, oval, oblong, triangular, rectangular, square, polygonal, irregular, uniform, non-uniform, variable and/or tapered. In some embodiments, spinal rod 28 may have various lengths. In some embodiments, the longitudinal element may include one or a plurality of tethers,

In some embodiments, the longitudinal element may have a flexible configuration and fabricated from materials, such as, for example, polyester, polyethylene, fabric, silicone, polyurethane, silicone-polyurethane copolymers, polymeric rubbers, polyolefin rubbers, elastomers, rubbers, thermoplastic elastomers, thermoset elastomers and elastomeric composites. In one embodiment, the flexibility of the longitudinal element includes movement in a lateral or side to side direction and prevents expanding and/or extension in an axial direction. In some embodiments, all or only a portion of the longitudinal element may have a semi-rigid, rigid or elastic configuration, and/or have elastic properties, such as the elastic properties corresponding to the material examples described above. In some embodiments, the longitudinal element may be compressible in an axial direction.

In assembly, operation and use, spinal implant system 10, similar to the systems described herein, is employed with a surgical procedure for treatment of a spinal disorder, such as those described herein, affecting a section of a spine of a patient. System 10 may also be employed with other surgical procedures. To treat the affected section of vertebrae V, body B of a patient is disposed in a lateral orientation, as shown in FIG. 1, relative to a surgical fixed surface, such as, for example, surgical table 32 configured for supporting body B.

Body B includes a lateral portion LP and a contra-lateral portion CLP. Portion CLP is disposed between portion LP and surgical table 32 when body B is disposed in the lateral orientation. In some embodiments, the components, such as, implants including bone fasteners, rods, interbody devices and plates, of spinal implant system 10 are delivered and implanted with body B while maintaining body B in the lateral orientation, not rotating body B from the lateral orientation and/or not moving body B into one or a plurality of alternate orientations relative to surgical table 32. This configuration and method of use of spinal implant system 10 reduces operating time for a surgical procedure and reduces radiation exposure due to fluoroscope or image guidance, for example, by eliminating procedural steps and patient repositioning by implanting system components in one body position.

In the lateral orientation, a medical practitioner obtains access to a surgical site including vertebral levels, such as, for example, vertebral levels V1, V2, V3, V4 of vertebrae V through a percutaneous surgical approach, whereby vertebrae V is accessed through a micro-incision, or sleeve that provides a protected passageway to the area. A plurality of percutaneous incisions, such as, for example, incisions I1, I2, I3 and I4 are made in tissue of portion CLP and disposed in a sagittal plane of body B substantially parallel to a sagittal plane SP of vertebrae V. In some embodiments, incisions I1, I2, I3 and I4 can be made and/or components of system 10 can be disposed with one or a plurality of sagittal planes oriented relative to plane SP, and/or oriented in an alternate plane of body B. The tissue adjacent incisions I1, I2, I3 and I4 are spaced apart to create and define a surgical pathway to the surgical site. Once access to the surgical site is obtained percutaneously, the components of spinal implant system 10 can be delivered or implanted with portion CLP.

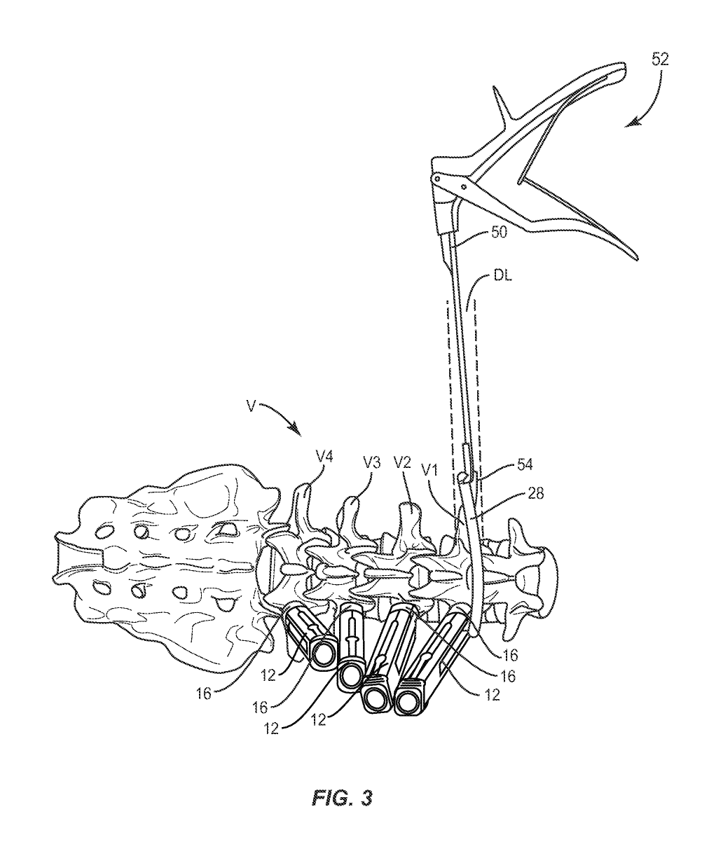

A pilot hole is made in each of vertebral levels V1, V2, V3 and V4 of portion CLP via incisions I1, I2, I3 and I4 through a pedicle of each of vertebral levels V1, V2, V3 and V4 along a surgical pathway. Fasteners 16, as described herein, are delivered through incisions I1, I2, I3 and I4 percutaneously to the surgical site. Fasteners 16 are each fastened via an extender 12 with vertebral levels V1, V2, V3 and V4 of vertebrae V, as shown in FIG. 3, in a uni-lateral orientation.

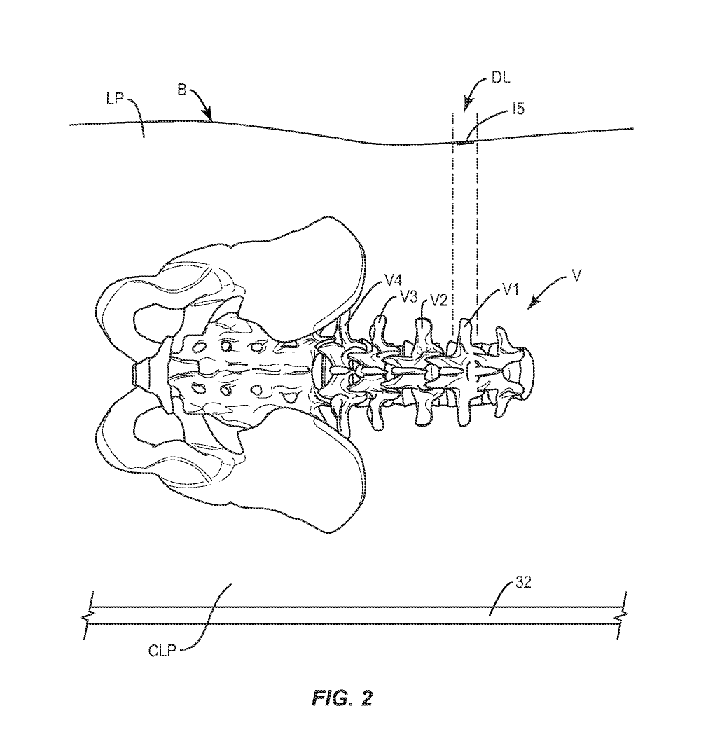

In the lateral orientation, the medical practitioner obtains access to the surgical site via a lateral incision 15 with body B, as shown in FIG. 2. The tissue adjacent incision 15 is spaced apart to create and define a surgical pathway DL to the surgical site, along a direct lateral surgical approach to the surgical site. Surgical pathway DL provides an opening or access path to vertebrae V. In some embodiments, incision 15 can be disposed at alternate orientations relative to sagittal plane SP and/or spinous process SPP, such as, for example, transverse and/or angular orientations.

A surgical instrument, such as, for example, a rod inserter or rod gripper 50 is engaged with spinal rod 28, as shown in FIG. 3. Rod gripper 50 includes a handle 52 connected with reciprocating distal end components 54 that are manipulated to capture spinal rod 28 in releasable engagement. Rod gripper 50 is manipulated such that spinal rod 28 is percutaneously delivered through incision 15 and along surgical pathway DL in a first orientation. In the first orientation, spinal rod 28 is disposed in alignment with surgical pathway DL and perpendicular to extenders 12, which are disposed in substantial alignment with the sagittal plane of body B having fasteners 16 disposed therein.

Handle 52 is manipulated via a squeezing motion or compression to relatively translate components 54 such that spinal rod 28 is rotated and/or flipped relative to rod gripper 50. Components 54 are spaced apart to engage portions of spinal rod 28 and pivot spinal rod 28 about the distal end of rod gripper 50.

Spinal rod 28 is rotated relative to the distal end of rod gripper 50 to a second orientation, as shown in FIG. 4, such that spinal rod 28 is oriented in alignment with the sagittal plane of body B having fasteners 16 disposed therein. In some embodiments, the second orientation includes spinal rod 28 being disposed in a parallel orientation relative to the sagittal plane of body B and/or sagittal plane SR In some embodiments, the second orientation includes spinal rod 28 being disposed in a co-planar orientation with the sagittal plane of body B. In some embodiments, the second orientation includes spinal rod 28 being disposed in a co-axial orientation with an axis defined by fasteners 16 and/or extenders 12,

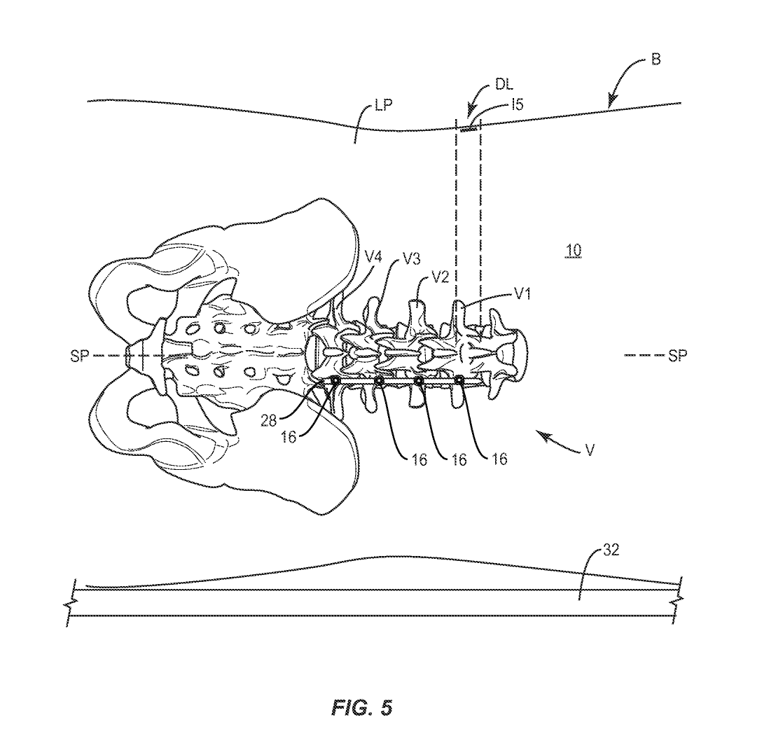

Rod gripper 50 rotates spinal rod 28 to the second orientation such that spinal rod 28 is disposed with extenders 12 for connection with fasteners 16. In the second orientation, rod gripper 50 is manipulated to insert spinal rod 28 with the U-shaped passageways of fasteners 16. Upon selected positioning of spinal rod 28 with the U-shaped passageways of fasteners 16, spinal rod 28 is secured with fasteners 16 via set screws, as shown in FIG. 5. In some embodiments, rods and/or fasteners are delivered to the surgical site and attached with lateral portion LP of vertebrae V, similar to that described herein with regard to portion CLP of vertebrae V. In some embodiments, fasteners are delivered to the surgical site, as described herein, and attached with vertebrae V in a bi-lateral orientation.

Upon completion of a procedure, as described herein, the surgical instruments, assemblies and non-implanted components of spinal implant system 10 are removed and the incisions are closed. One or more of the components of spinal implant system 10 can be made of radiolucent materials such as polymers. Radiomarkers may be included for identification under x-ray, fluoroscopy, CT or other imaging techniques. In some embodiments, the use of surgical navigation, microsurgical and image guided technologies may be employed to access, view and repair spinal deterioration or damage, with the aid of spinal implant system 10. In some embodiments, spinal implant system 10 may include one or a plurality of plates, connectors and/or bone fasteners for use with a single vertebral level or a plurality of vertebral levels.

In one embodiment, spinal implant system 10 includes an agent, which may be disposed, packed, coated or layered within, on or about the components and/or surfaces of spinal implant system 10. In some embodiments, the agent may include bone growth promoting material, such as, for example, bone graft to enhance fixation of the components and/or surfaces of spinal implant system 10 with vertebrae V. In some embodiments, the agent may include one or a plurality of therapeutic agents and/or pharmacological agents for release, including sustained release, to treat, for example, pain, inflammation and degeneration.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A method for treating a spine, the method comprising the steps of:
disposing a body including vertebrae in a lateral orientation relative to a surgical fixed surface configured for supporting the body;
delivering a plurality of first implants adjacent the vertebrae such that the plurality of first implants are disposed along a sagittal plane of the body;
attaching at least one surgical instrument to the plurality of first implants;
creating an incision in tissue of the body;
spacing the tissue adjacent the incision to define a surgical pathway to the vertebrae along a lateral surgical approach;
delivering a second implant along the surgical pathway in a first orientation such that the second implant is aligned with the surgical pathway; and
rotating the second implant to a second orientation such that the second implant is aligned with the sagittal plane.

2. A method as recited in claim 1, wherein in the first orientation the second implant is perpendicular to the at least one surgical instrument.

3. A method as recited in claim 1, wherein in the first orientation the second implant is perpendicular to the sagittal plane.

4. A method as recited in claim 1, wherein in the second orientation the second implant is parallel with the at least one surgical instrument.

5. A method as recited in claim 1, wherein the incision is created along a substantially transverse plane of the body.

6. A method as recited in claim 1, wherein spacing the tissue includes spacing tissue along the sagittal plane.

7. A method as recited in claim 1, wherein the step of delivering the plurality of first implants includes percutaneously delivering the plurality of first implants.

8. A method as recited in claim 1, wherein the step of delivering the second implant includes percutaneously delivering the second implant.

9. A method as recited in claim 1, wherein the plurality of first implants includes bone screws.

10. A method as recited in claim 1, wherein the surgical instrument includes an extender.

11. A method as recited in claim 1, wherein the surgical instrument includes a plurality of extenders.

12. A method as recited in claim 1, wherein the second implant includes a spinal rod.

13. A method as recited in claim 1, wherein the second implant is engaged with an inserter for insertion and manipulation along the surgical pathway between the first orientation and the second orientation.

14. A method as recited in claim 13, wherein the inserter is configured to rotate the second implant from the first orientation such that the second implant is perpendicular to the surgical instrument and the second orientation such that the second implant is parallel with the surgical instrument.

15. A method for treating a spine, the method comprising the steps of:
disposing a body including vertebrae in a lateral orientation relative to a surgical fixed surface configured for supporting the body;
delivering a plurality of bone screws via the surgical pathway adjacent the vertebrae such that the plurality of bone screws are disposed along a sagittal plane of the body;
attaching an extender to each of the plurality of bone screw;
creating an incision in tissue of the body;
spacing the tissue with a surgical instrument to define a surgical pathway to the vertebrae along a lateral surgical approach;
delivering a spinal rod along the surgical pathway in a first orientation such that the spinal rod is aligned with the surgical pathway; and
rotating the spinal rod to a second orientation such that the spinal rod is aligned with the sagittal plane.

16. A method as recited in claim 15, wherein in the first orientation the spinal rod implant is perpendicular to the extenders.

17. A method as recited in claim 15, wherein in the second orientation the spinal rod is parallel with the extenders.

18. A method as recited in claim 15, wherein the step of delivering the plurality of bone screw includes percutaneously delivering the plurality of bone screw.

19. A method as recited in claim 1, wherein the step of delivering the spinal rod includes percutaneously delivering the spinal rod.

20. A method for treating a spine, the method comprising the steps of:
disposing a body including vertebrae in a lateral orientation relative to a surgical fixed surface configured for supporting the body;
percutaneously delivering a plurality of first implants along adjacent the vertebrae such that the plurality of first implants are disposed along a sagittal plane of the body;
attaching at least one surgical instrument o the plurality of first implants;
creating an incision in tissue of the body along a substantially transverse plane of the body;
spacing the tissue adjacent the incision to define a surgical pathway to the vertebrae along a lateral surgical approach;
percutaneously delivering a second implant via the surgical pathway in a first orientation such that the second implant is aligned with the surgical pathway; and
rotating the second implant to a second orientation such that the second implant is aligned with the sagittal plane.
